(19) United States
(12) Patent Application Publication
(10) Pub. No.: US 2009/0197320 A1
(43) Pub. Date: Aug. 6, 2009

(54) ANCESTRAL DENGUE VIRUS ENVELOPE PROTEIN

(75) Inventor: Xiaofeng Fan, St. Louis, MO (US)

Correspondence Address: HUSCH BLACKWELL SANDERS LLP, 720 OLIVE STREET, SUITE 2400, ST. LOUIS, MO, 63101, US

(73) Assignee: SAINT LOUIS UNIVERSITY, St. Louis, MO (US)

(21) Appl. No.: 12/067,018

(22) PCT Filed: Sep. 15, 2006
(86) PCT No.: PCT/US06/36107

§ 371 Date: Aug. 29, 2008

Related U.S. Application Data
(60) Provisional application No. 60/718,178, filed on Sep. 16, 2005.

(51) Int. Cl. C12N 7/01; C12N 15/40

(52) U.S. Cl. 435/235.1; 536/23.72

(57) ABSTRACT

Disclosed is a dengue virus envelope protein sequence derived via ascertainment of a most recent common ancestor of the three dengue serotype variants, DENV-1, DENV-2, DENV-3 and DENV-4. This synthetic dengue virus envelope protein can be used as a tetravalent vaccine in the prevention of dengue fever, dengue hemorrhagic fever and dengue septic shock.

### GOVERNMENTAL SUPPORT

This work was supported by the U.S. Department of Health and Human Services/National Institutes of Health grant. The U.S. Government has certain rights in this invention.

### SEQUENCE LISTING

A written (on paper) sequence listing is appended below and computer readable form of the sequence listing is included, both of which are herein incorporated by reference. Applicant hereby states that the information recorded in computer readable form is identical to the written (on paper) sequence listing.

### BACKGROUND OF THE INVENTION

1. Field of the Invention

The invention is directed to subunit vaccines in general, and a dengue virus envelope protein vaccine in particular.

2. Description of the Related Art

Dengue Virus

The dengue viruses are members of the Flaviviridae family. Dengue consists of four antigenically related but distinct serotypes, designated DENV-1, DENV-2, DENV-3, and DENV-4. They have a single-stranded RNA genome that contains an 11-kb plus-sensed RNA genome that is composed of seven nonstructural protein genes and three structural protein genes: core (C, 100 amino acids), membrane (M, 75 amino acids), and envelope (E, 595 amino acids). The domains responsible for neutralization, fusion, and interactions with virus receptors are associated with the envelope protein.

The dengue viruses are transmitted to humans by the bite of infective female mosquitoes of the genus Aedes (primarily the species aegypti, but also albopictus and polynesienses also are involved). The virus manifests itself into 3 types of illnesses: Dengue fever (DF), Dengue hemorrhagic fever (DHF), and Dengue Septic Shock (DSS). DF is a severe, flu-like illness that affects infants, children, adolescents, and adults. Its incubation period after the mosquito bite occurs is between 3 and 8 days. Although it may be incapacitating, the prognosis for a DF patient is favorable and generally recovery occurs after having 7 to 10 days of illness. DHF is an acute illness with hemorrhagic manifestations, which, if it becomes critical, may result in Dengue Septic Shock (DSS). Death can occur within 12 to 24 hours, or the patient may recover quickly after receiving appropriate therapy.

Among the factors implicated in the resurgence of the dengue virus globally are failures to control the Aedes population, increased air travel to and from endemic areas, uncontrolled urbanization, unprecedented population growth, along with other features such as El Niño. The control or prevention of dengue fever and DHF involves combating the vector mosquitoes, implementing good surveillance systems, and developing effective vaccines.

Major epidemics of dengue-like illnesses have been reported globally as far back as the latter part of the eighteenth century. The first recorded epidemic of dengue-like disease dates back to 1779 to 1780. In the eighteenth and early nineteenth centuries, epidemics or regional pandemics of dengue fever occurred approximately every 10 to 40 years in tropical regions of the world. During the later nineteenth and early twentieth centuries, epidemics of dengue or DHF raged through countries in southeast Asia approximately every 3 to 5 years. During World War II, the movement of troops provided the virus with a large supply of new susceptible hosts on a continuous basis, thereby increasing the spread of disease in southeast Asia. Subsequent movements of those hosts or war machinery or both facilitated the circulation of virus serotypes throughout the region and fostered hyperendemicity. During the post-World War II era, millions of people moved after the war from the poor rural countryside to the cities. These postwar conditions led to both a tremendous increase in the incidence of dengue and the emergence of DHF, which was discovered in Manila in 1953. Since 1953, when the first epidemic occurred in the Philippine Islands (1953-1954), DHF has increased considerably in frequency, geographical scope, and number of cases. During the middle and later twentieth century, large increases in unplanned urbanization (resulting in large populations living in high-density areas with inadequate systems of water and solid waste management) provided excellent breeding grounds for mosquitoes and contributed to a significant increase in the incidence of dengue fever and the emergence of DHF as a major public health problem. Until 1970, only nine countries in the world had experienced epidemics of DHF, but by 1995, the number had increased more than four-fold and included the first major epidemic, which occurred in Cuba in 1981. During the 1950s, the average annual number of DHF cases had been 908, but by the period from 1990 to 1998 that average had increased to 514,139 cases. In 1981, DHF emerged in the Americas, and it emerged in 1989 in Sri Lanka, along with the appearance there of a new dengue virus serotype 3 (DENV-3), subtype III variant.

One of the largest pandemics occurred between 1997 and 1998. In 1997, Malaysia recorded a record 19,544 cases of dengue, representing a 37.4 percent higher number than the highest number previously reported since 1973. Despite remedial efforts made in other countries, a pandemic occurred, during which more than 1.2 million cases of dengue fever and DHF were reported to the WHO from 56 countries. Unprecedented global epidemic activity of dengue was noted in the American hemisphere in 2001, where more than 609,000 cases of dengue were reported, representing more than double the number recorded in the same region in 1995. Today, the disease is endemic in more than 100 countries in Africa, the Americas, the Eastern Mediterranean, southeast Asia, and the western Pacific, the latter two being the areas most seriously affected.

Dengue virus is the agent responsible for an important arbovirus disease, with an estimated annual infection rate in excess of 50 million (38). The spectrum of illness ranges from unapparent, mild disease to the severe and occasionally fatal clinical diseases, dengue hemorrhagic fever (DHF) and dengue shock syndrome (DSS). The pathogenesis of DHF and DSS remains elusive. Although other factors such as viral virulence and host characteristics are of importance, there is compelling evidence from clinical and experimental studies that secondary infection is the main risk factor for DHF (15). Primary infection with one of dengue virus serotypes (recall that there are at least four known Dengue Virus serotypes) provides lifelong homologous immunity with only transient cross-protection against the remaining three serotypes (20). The pre-circulating anti-dengue antibodies acquired during primary infection with a different serotype form complexes with dengue viruses, which infect mononuclear phagocytes with enhanced efficiency and as a consequence a higher number of cells are infected, a phenomenon known as antibody-dependent enhancement (ADE) (15).

ADE has been demonstrated with non-neutralizing antibodies against dengue virus envelope protein and even sub-neutralizing cross-reactive antibodies (24).

Presently, there is no licensed vaccine for dengue virus. Due to the potential for infection with four serotypes and no cross-serotype immunity, an effective dengue vaccine must induce strong protective responses against all four dengue serotypes for a sustained period. For this reason, a tetravalent rather than a monovalent dengue vaccine has been suggested. Various approaches have been tried for dengue vaccine development, including inactivated whole virus, live-attenuated virus, chimeric virus, subunit vaccine and DNA vaccine. However, low immunogenicity is often found for attenuated virus, chimeric virus and subunit vaccines. Attenuated dengue isolates may return to pathogenic isolates due to genetic instability or through recombination (18). Viral interference and neurovirulence are also concerns. A DNA vaccine may result in stronger immunogenicity due to the high-level intracellular expression of foreign genes. However, there are some critical unresolved issues, such as potential oncogenesis. More importantly, any approach using the tetravalent vaccination strategy, always results in an immune bias in which neutralizing antibodies are missing to at least one of four dengue serotypes (reviewed in references 3 and 4).

### CITED REFERENCES

The following numbered references are cited throughout this disclosure. The references are used to support and illustrate the disclosure, and thus are hereby incorporated by references. However, Applicant reserves the right to challenge the veracity of any statements made in these references.
(1) Andre S, Seed B, Eberle J, Schraut W, Bultmann A, Haas J. Increased immune response elicited by DNA vaccination with a synthetic gp120 sequence with optimized codon usage. J Virol 1998; 72:1497-503.
(2) Belinda S, Chang W, Jensson K, Kazmi M A, Donoghue M J and Sakmar T P. Recreating a functional ancestral Archosaur visual pigment. Mol Biol Evol 2002; 19:1483-89.
(3) Chang G J, Kuno G, Purdy D E and Davis B S. Recent advancement in flavivirus vaccine development. Expert Review of Vaccines 2004; 3:199-220.
(4) Eckels K H, and Putnak R. Formalin-inactivated whole virus and recombinant subunit flavivirus vaccines. Advances in Virus Research 2003; 61:395-418.
(5) Ellington A and Cherry J M. 1997. Characteristics of amino acids. A.1C.1-A.1C.12. In F. M. Ausubel, R. Brent, R. E. Kingston, D. D. Moore, J. G. Seidman, J. A. Smith, and K. Struhl (ed.), Current protocols in Molecular Biology. John Wiley & Sons, Inc., New York, N.Y.
(6) Fan X and Di Bisceglie A M. Derivation, origin-dating and assembly of ancestral hepatitis C virus (HCV) envelope sequences. Hepatology 2002; 36:203A.
(7) Fan X, Lang D M, Xu Y, Lyra A C, Yusim K, Everhart J E, Korber B T, Perelson A S and Di Bisceglie A M. Liver transplantation with hepatitis C virus-infected graft: Interaction between donor and recipient viral strains. Hepatology 2003; 38:25-33.
(8) Felsenstein J. Phylogenies from molecular sequences: inference and reliability. Annu Rev Genet 1988; 22:521-65.
(9) Gao F, Weaver E A, Lu Z, Li Y, Liao H X, Ma B, Alam S M, Scearce R M, Sutherland L L, Yu J S, Decker J M, Shaw G M, Montefiori D C, Korber B T, Hahn B H and Haynes B F. Antigenicity and immunogenicity of a synthetic human immunodeficiency virus type 1 group m consensus envelope glycoprotein. J Virol 2005; 79:1154-63.
(10) Gaschen B, Taylor J, Yusim K, Foley B, Gao F, Lang D, et al. Diversity considerations in HIV-1 vaccine selection. Science 2002; 296:2354-60.
(11) Goldman N. Statistical tests of models of DNA substitution. J Mol Evol 1993; 36:182-98.
(12) Graham S W, Olmstead R G and Barrett S C H. Rooting phylogenetic trees with distant outgroups: a case study from the commelinoid monocots. Mol Biol Evol 2002; 19:1769-81.
(13) Grote A, Hiller K, Scheer M, Munch R, Nortemann B, Hempel D C and Jahn D. JCat: a novel tool to adapt codon usage of a target gene to its potential expression host. Nucleic Acid Res 2005; 33:W526-31.
(14) Guindon S and Gascuel O. A simple, fast, and accurate algorithm to estimate large phylogenies by maximum likelihood. Systematic Biology 2003; 52:696-704.
(15) Halstead S. Pathophysiology and pathogenesis of dengue hemorrhagic fever. In Thongcharoen P, ed. Monograph on Dengue/Dengue Hemorrhagic Fever. WHO Regional Publication, SEARO no 22, 1993:80-103.
(16) Higgins D G and Sharp P M. CLUSTAL: a package for performing multiple sequence alignment on a microcomputer. Gene 1988; 73:237-44.
(17) Holmes E C and Twiddy S S. The origin, emergence and evolutionary genetics of dengue virus. Infection, Genetics and Evolution 2003; 3:19-28.
(18) Holmes E C, Worobey M and Rambaut A. Phylogenetic evidence for recombination in dengue virus. Mol Biol Evol 1999; 16:405-9.
(19) Huelsenbeck J P, Bollback J P and Levine A M. Inferring the root of a phylogenetic tree. Syst Biol 2002; 51:32-43.
(20) Innis B L. Antibody responses to dengue virus infection. In D. J. Gubler and G. Kuno (ed.), Dengue and Dengue hemorrhagic fever. CAB International, Wallingford, United Kingdom. 1997:221-243.
(21) Kumar S, Tamura K, Jakobsen I B and Nei M. MEGA2: Molecular evolutionary genetics analysis software. Bioinformatics 2001; 17:1244-5.
(22) Jermann T M, Opitz J G, Stackhouse J and Benner S A. Reconstructing the evolutionary history of the artiodactyl ribonuclease superfamily. Nature 1995; 374:57-9.
(23) Lole K S, Bollinger R C, Paranjape R S, Gadkari D, Kulkarni S S, Novak N G, Ingersoll R, Sheppard H W, Ray S C. Full-length human immunodeficiency virus type 1 genomes from subtype C-infected seroconverters in India, with evidence of intersubtype recombination. J Virol 1999; 73:152-60.
(24) Morens D M. Antibody-dependent enhancement of infection and the pathogenesis of viral disease. Clin Infect Dis 1994; 19:500-12.
(25) Novella I S, Zarate S, Metzgar D, Ebendick-Corpus B E. Positive selection of synonymous mutations in vesicular stomatitis virus. J Mol Biol 2004; 342:1415-21.
(26) Posada D and Crandall K A. Modeltest: testing the model of DNA substitution. Bioinformatics 1998; 14:817-8.
(27) Posada D and Crandall K A. Selecting the best-fit model of nucleotide substitution. Syst Biol 2001; 50:580-601
(28) Sharp P M and Li W H. The codon adaptation index, a measure of directional synonymous codon usage bias and its potential applications. Nucleic Acid Res. 1987; 15:1281-95.
(29) Swofford D L. PAUP*: Phylogenetic Analysis using Parsimony and Other Methods. Version 4.02b. Sinauer Associates. Sunderland, Mass.
(30) Tamura K and Nei M. Estimation of the number of nucleotide substitutions in the control region of mitochondrial DNA in humans and chimpanzees. Mol Biol Evol 1993; 10:512-26.
(31) Tolou H J G, Couissinier-Paris P, Durand J P, Mercier V, de Pina J J, de Micco P, Billoir F, Charrel R N and de Lamballerie X. Evidence for recombination in natural populations of dengue virus type 1 based on the analysis of complete genome sequences. J Gen Virol 2001; 82:1283-90.
(32) Twiddy S S and Holmes E C. The extent of homologous recombination in members of the genus Flavivirus. J Gen Virol 2003; 84:429-440.
(33) Twiddy S S, Holmes E C and Rambaut A. Inferring the rate and time-scale of dengue virus evolution. Mol Biol Evol 2003; 20:122-9.
(34) Twiddy S S, Woelk C H and Holmes E C. Phylogenetic evidence for adaptive evolution of dengue virus in nature. J Gen Virol 2002; 83:1679-89.
(35) Uzcategui N Y, Camacho D, Cmach G, Cuello de Uzcategui R, Holmes E C and Gould E A. Molecular epidemiology of dengue type 2 virus in Venezuela: evidence for in situ virus evolution and recombination. J Gen Virol 2001; 82:2945-53.
(36) Whalen R G, Kaiwar R, Soong N W and Punnonen J. DNA shuffling and vaccine. Curr Opin Mol Ther 2001; 3:31-6.
(37) Wheeler W C. Nucleic acid sequence phylogeny and random outgroups. Cladistics 1990; 6:363-8.
(38) WHO (2000). Strengthening implementation of the global strategy for dengue fever/dengue hemorrhagic fever prevention and control: Report of the informal consultation (http://www.who.int/emc-documents/dengue/whoedsdenic20001c.html)
(39) Wisconsin GCG package. Version 10.0. Oxford Molecular Group, Inc.
(40) Worobey M, Rambaut A and Holmes E C. Widespread intra-serotype recombination in natural populations of dengue virus. Proc Natl Acad Sci USA 1999; 96:7352-7.
(41) Yang Z. Estimating the pattern of nucleotide substitution. J Mol Evol 1994; 39:105-11.
(42) Yang Z. PAML: A program package for phylogenetic analysis by maximum likelihood. Com Appl Biosci 1997; 13:555-6.
(43) Kelly E P. Greene J J. King A D. Innis B L. Purified dengue 2 virus envelope glycoprotein aggregates produced by baculovirus are immunogenic in mice. Vaccine 2000; 18:2549-59.
(44) Wu S C, Lin Y J and Yu C H. Baculovirus-insert cell expression, purification, and immunological studies of the full-length Japanese encephalitis virus envelope protein. Enzyme and Microbial technology 2003; 33: 438-44.

### SUMMARY OF THE INVENTION

The inventor has derived certain polynucleotide and polypeptide sequences, which represent conceptual ancestral and consensus sequences of the envelope proteins of at least the four major dengue virus serotypes DENV1, DENV2, DENV3 and DENV4. The inventor envisions that any one or more of the sequences can be used as an effective tetravalent vaccine directed against all four major serotypes of dengue virus.

In one embodiment, the invention is directed to a conceptually derived ancestral dengue virus envelope protein polynucleotide, which represents a hypothetical ancestor for at least the four dengue serotypes, DENV1, DENV2, DENV3 and DENV4. Conceptually derived ancestral dengue virus envelope protein polynucleotides include those sequences containing sequences as set forth in SEQ ID NOs:1 through 11. A preferred ancestral dengue virus envelope protein polynucleotide has a sequence that is at least 81% identical to SEQ ID NO:1 or SEQ ID NO:3, or at least 88% identical to SEQ ID NO:2. A more preferred ancestral dengue virus envelope protein polynucleotide has a sequence that is set forth in any one of SEQ ID NO:1 through SEQ ID NO:3. A most preferred ancestral dengue virus envelope protein polynucleotide has a sequence that is set forth in SEQ ID NO:2.

In another embodiment, the invention is directed to a conceptually derived ancestral dengue virus envelope polypeptide, which represents a hypothetical ancestor for at least the four dengue serotypes, DENV1, DENV2, DENV3 and DENV4. Conceptually derived ancestral dengue virus envelope polypeptides include those sequences containing sequences as set forth in SEQ ID NOs:12 through 21. A preferred ancestral dengue virus envelope polypeptide has a sequence that is at least 84% identical to SEQ ID NO:12 or is at least 82% identical to SEQ ID NO:13. A more preferred ancestral dengue virus envelope polypeptide has a sequence that is at least 88% identical to SEQ ID NO:12 and 13. A most preferred ancestral dengue virus envelope polypeptide has a sequence that is set forth in SEQ ID NO:12 or SEQ ID NO:13.

In yet another embodiment, the invention is directed to a method for developing an ancestral nucleotide sequence through reconstruction of phylogenetic trees. The ancestral nucleotide sequence may be directed to any one of myriad viruses or virus families. Preferred viruses are linear, single stranded RNA viruses. More preferred viruses are flaviviruses. Most preferred viruses are viruses of the dengue group. The method involves the steps of retrieving virus nucleic acid sequences from a genetic database (e.g., GenBank) and then editing and aligning those sequences using editing and alignment programs, which include for example Clustal W (ref. 16), the BioEdit program available from North Carolina State University (available at http://www.mbio.ncsu.edu/BioEdit/bioedit.html), and the SegEd program available in the GCG package (ref. 39). Any missing information may be determined by phylogenetic analyses, such as for example Molecular Evolutionary Genetics Analysis (MEGA; see ref. 21). The sequences are then filtered to remove sequences that are below a particular size cut-off, e.g., in the case of a dengue envelope protein nucleic acid sequence, the cut-off is about less than about 1485 or 1479 nucleotides. Those remaining sequences that show signs of recombination are eliminated. The now remaining sequences are subjected to split decomposition analysis to remove any phylogenetic noise (see ref. 7). The now remaining sequences having greater than 99% identity at the nucleotide level are reduced to a single representative sequence. Model simulation and phylogenetic reconstruction are applied to the now remaining sequences. A preferred model is a hierarchical likelihood ratio test (hLRT) simulated with the program Modeltest (see refs. 26 and 27). A phylogenetic tree is then constructed by heuristic search using a maximum likelihood (ML) approach for separate or combined virus serotypes. ML trees can be constructed using any one or more of known programs (e.g., PAUP, PHYML, see refs. 14 and 29.) Once the trees are produced, the tree may be rooted using a strict or relaxed molecular clock model (see refs. 33 and 34), non-reversible models of substitution, midpoint rooting, and/or outgroup criterion. (see refs. 12, 19, 33, 34, 37 and 41). The correctly rooted tree is then used as a template to simulate an ancestral sequence. Simulation of ancestral sequences at each internal node as well as the most recent common ancestor (MRCA) are inferred using a reconstruction program, such as for example the baseml program of the PALM package (see ref. 42). The ancestral sequence(s) are reconstructed at the nucleotide level.

In yet another embodiment, the invention is directed to a vaccine comprising an ancestral dengue sequence (supra), wherein the vaccine protects a recipient of the vaccine against all four major serotypes of dengue virus. In another embodiment, the invention is directed to an immune system stimulating composition comprising an ancestral dengue sequence (supra), wherein the composition elicits an immune reaction in the recipient against all four major serotypes of dengue virus.

### BRIEF DESCRIPTION OF THE DRAWINGS

FIG. 1 depicts the evolutionary relationship among nine current dengue virus sequences is established by the construction of a phylogenetic tree that connects nine dengue envelope sequences through a common node, i.e., the most recent common ancestor (MRCA).

FIG. 2 depicts the processing of dengue sequence data, starting with 2015 dengue sequences were retrieved from GenBank, which were subsequently filtered to identify 189 dengue sequences, which were used for model simulation and phylogenetic reconstruction.

FIG. 3 depicts the maximum likelihood reconstruction with 189 full-length dengue virus envelope sequences. All possible genotypes within each serotype are indicated. Bootstrap test was done with 100 replicates as shown at major branches. The tree was rooted by applying molecular clock. The node at which the ancestral sequence will be inferred is also indicated. MRCA, most recent common ancestor.

FIG. 4 depicts a similarity plot of DengueA1 (SEQ ID NO:1) to consensus sequences of each wild-type dengue serotypes.

FIG. 5 depicts a similarity polt of DengueA2 (SEQ ID NO:2) to consensus sequences of each wild-type dengue serotype.

FIG. 6 depicts a similarity polt of DengueC (SEQ ID NO:3) to consensus sequences of each wild-type dengue serotype.

FIG. 7 depicts the expression of codon optimized ancestral Dengue DA4 and wild-type Dengue DW1 in sf9 cells by Western blot analysis. The sf9 cells were transfected with pBacPAk9-DA4-H6 and pBacPAK9-DW1-H6 respectively. Forty-eight hours post transfection, the supernatant was collected and served as primary recombinant virus. Cell lysates were applied for Western blotting analysis by using monoclonal anti-His6 antibody (Qiagen) as primary antibody. For the positive control lane, 10 ng of purified H6 tagged GST-IkB-H6 protein was loaded. For other lanes, 20 ug of cell lysate was loaded on each lane.

FIG. 8 depicts virus titration for dengue core protein production. After the third round amplification, recombinant baculovirus was added to sf9 cells as indicated (infra). Forty-eight hours after viral infection, cells were collected and lysed, followed by Western blotting analysis. The maximum yield of recombinant protein after optimization is about 20 ug/L for DA4 and 10 ug/L for DW1.

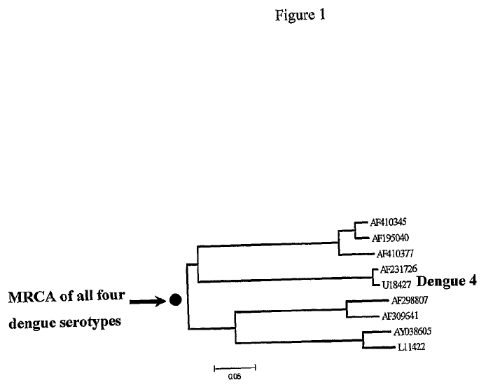

### DETAILED DESCRIPTION OF SEVERAL PREFERRED EMBODIMENTS

The following description is merely meant to illustrate, but not limit, the invention.

### Example 1

### Inference of Ancestral Dengue Envelope Sequences

Data compilation: A total of 2015 sequences regarding dengue virus were retrieved from the GenBank (http://www.ncbi.nlm.nih.gov/Genbank/index.html). Each sequence was manually examined to determine its serotype, genome location and length. This resulted in a collection of 712 sequences containing the dengue envelope gene. Sequences were edited and aligned with Clustal W (16), BioEdit (http://www.mbio.ncsu.edu/BioEdit/bioedit.html) and SeqEd program in GCG package (39). Missing serotype/genotype information for some sequences were determined by phylogenetic analyses with MEGA (Molecular Evolutionary Genetics Analysis) (21) under neighbor-joining approach with kumar-2 parameter as nucleotide substitution model. We then filtered the data by excluding sequences that meet one of following criteria:

a) Not full-length dengue envelope gene, i.e., less than 1485 bp for dengue serotypes 1, 2 and 4 and less than 1479 bp for dengue serotype 3.

b) Recombinants. At the genetic level, mutation, recombination and reassortment are three major events driving the evolution of a given microbe. Unlike mutation, recombination frequently results in evolutionary “jump”. Since all current approaches for phylogenetic reconstruction assume that evolution is solely contributed by mutation and selection, the inclusion of recombinants will generate phylogenetic noise that interferes with the reconstruction of correct tree topologies. We therefore excluded all isolates that showed phylogenetic evidence for genetic recombination (Table 1). For the remaining data set, split decomposition analysis was conducted to see any possible phylogenetic noise as we previously described (7): this was not detected as shown by a bifurcating tree without any network among isolates (data not shown).

TABLE 1
Dengue isolates with phylogenetic evidence
of recombination in envelope domain.

| Serotype | Gene name | GenBank accession # | Reference |
|---|---|---|---|
| Den-1 | Philippines84-162 | D00503 | 40 |
| | Thailand80-AHF82-80 | D00502 | 40 |
| | French Guiana-FGA/89 | AF226687 | 18 |
| | Brazil-BR/90 | S64849 | 18 |
| | S275/90 | E06832 | 31 |
| Den-2 | D80-038 | M24448 | 32 |
| | MalaysiaM3-M3 | X15214/X17340 | 40 |
| | Malaysia68-P7-863 | U89517 | 40 |
| | Mara4 | AF100466 | 35 |
| Den-3 | Tahiti65-2167 | L11619 | 40 |
| | Puerto Rico77-1340 | L11434 | 40 |
| | Mozambique85-1558 | L11430 | 40 |
| Den-4 | H241-P | S66064 | 32 |
| | Indonesia73-30153 | U18428 | 40 |

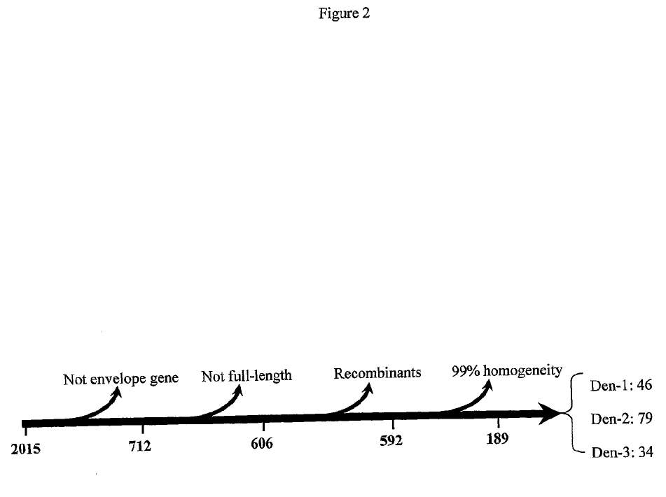

Many dengue sequences were deposited to GenBank as a group of isolates from a given geographical area. These sequences show extreme genetic homogeneity. Based on previous experience, exclusion of those sequences has no effect on final phylogenetic topologies. However, inclusion of those sequences will dramatically increase computation time. Therefore, for sequences showing more than 99% genetic homogeneity at the nucleotide level, only one of them was included. This was done by generating a large nucleotide distance matrix for each dengue serotype with MEGA (21). Based on these matrixes, we excluded sequences with less than 15 nucleotide difference (˜1%) over the entire dengue envelope gene (FIG. 2). Finally, 189 dengue envelope sequences were selected and used for model simulation and phylogenetic reconstruction.

All phylogenetic methods make assumptions, whether explicit or implicit, about the process of DNA substitution (8). An unsuitable model will result in erroneous phylogenetic reconstruction that misrepresents the interpretation of evolutionary history. In this project, it was especially important to explore a model that fits the data best because of a direct relationship between the model chosen and the inference of ancestral sequences. Therefore, each dengue data set was estimated for best-fit models by hierarchical likelihood ratio tests (hLRTs) that were simulated with the program Modeltest which may test 56 evolutionary models (26, 27). As shown in Table 2, all dengue serotypes, either separate or combined, follow a similar nucleotide substitution model, TrN. This intrinsic stability in nucleotide substitution strengthens the feasibility to pursue their evolutionary ancestor.

TABLE 2
Results of hierarchical likelihood ratio tests for different
models of sequence evolution in dengue virus.

| | | | Base frequencies | | | | | | Model |
|---|---|---|---|---|---|---|---|---|---|
| Data | No. | K | A | C | G | T | I | α | Selected |
| Den-1 | 46 | 6 | 0.3265 | 0.2089 | 0.2513 | 0.2134 | 0 | 0.3056 | TrN + G |
| Den-2 | 79 | 7 | 0.3447 | 0.2126 | 0.2363 | 0.2063 | 0.2785 | 0.7971 | TrN + I + G |
| Den-3 | 34 | 6 | 0.3171 | 0.2017 | 0.2660 | 0.2152 | 0 | 0.2499 | TrN + G |
| Den-4 | 30 | 7 | 0.3143 | 0.1977 | 0.2667 | 0.2213 | 0.5111 | 1.1130 | TrN + I + G |
| Den-all | 189 | 7 | 0.3581 | 0.2235 | 0.2226 | 0.1958 | 0.1624 | 0.6853 | TrN + I + G |

K, number of free parameters; I, proportion of invariable sites; α, shape parameter of gamma distribution; G, variable sites. TrN, a nucleotide substitution model developed by Tamura and Nei (30).

The best trees were recovered by heuristic search using maximum likelihood (ML) approach for separate or combined dengue serotypes. The best-fit model and relative parameters described in Table 2 were applied. All processes were completed with the program PAUP* (29). For data sets Den-2 (n=79) and Den-all (n=189), ML trees cannot be constructed with PAUP* due to the large numbers of sequences that assume unaffordable computation (years). We therefore produced ML trees with PHYML program that implanted a simple hill-climbing algorithm for heuristic tree search and used a distance-based tree as a starting point (14). The trees produced by PHYML were then transferred into PAUP* for further optimization and rooting by either outgroup or molecular clock approach (see section B4).

Molecular clock hypothesis was examined for all data sets by using the likelihood ratio test (LRT) (11). The log-likelihood values were scored with PAUP* for all ML trees built with or without the involvement of molecular clock assumption. The significance was determined by one-way Chi Square test. Molecular clock hypothesis was rejected by all data sets (Table 3).

TABLE 3
Likelihood ratio test (LRT) of the molecular clock hypothesis.

| | Den-1 | Den-2 | Den-3 | Den-4 | Den-All |
|---|---|---|---|---|---|
| Number | 46 | 79 | 34 | 30 | 189 |
| -ln L (Clock) | 8313.61065 | 13499.47781 | 6069.26427 | 6024.62043 | 31089.69478 |
| -ln L (No clock) | 8241.94460 | 13251.58066 | 5951.51049 | 5950.91177 | 30526.98231 |
| δ | 143.34 | 495.8 | 235.5 | 147.42 | 1125.42 |
| P value | <0.0001 | <0.0001 | <0.0001 | <0.0001 | <0.0001 |
| Molecular Clock | Rejected | Rejected | Rejected | Rejected | Rejected |

The degree of freedom (df) is equal to the number of the taxa minus 2 and δ is equal to the difference in log-likelihood scores multiplied by 2.

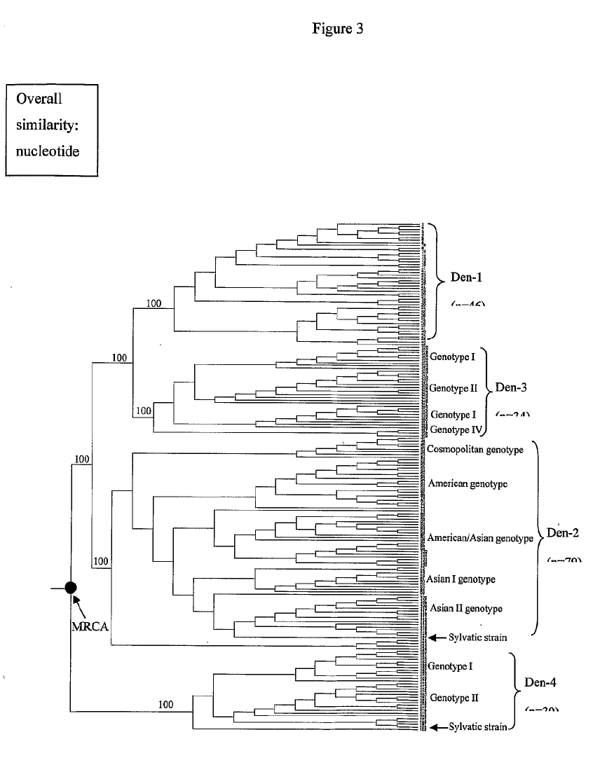

The root of a phylogenetic tree represents its first and deepest split, and it therefore provides the crucial time point for polarizing the historical sequences of all subsequent evolutionary events. An incorrectly rooted tree can result in profoundly misleading inferences of taxonomic relationships and character evolution. After the determination of a suitable evolutionary model, it is mandatory to root the tree to generate a correct topology, which will serve as the template for the inference of ancestral sequences. There are several methods available for rooting phylogenetic trees, including non-reversible models of substitution, midpoint rooting, the outgroup criterion and the molecular clock (12, 19). The first two approaches have been proven to be problematic (37, 41). Although outgroup criterion has been frequently used in phylogenetic practice, there is no well-identified virus as an outgroup to root the tree constructed with all four dengue serotypes. As expected with any real sequence data, the molecular clock hypothesis is rejected for dengue virus, indicating an unequal evolutionary rate among dengue isolates (Table 3). However, the method we used to test the molecular clock hypothesis did not consider time points at which sequences were isolated, referred as a “strict” molecular clock model. When considering the time scale, the evolution of dengue virus shows a molecular clock pattern, referred as a “relaxed” clock model (33, 34). Additionally, the root was more consistently identified with molecular clock assumption comparing to the outgroup approach where either Yellow Fever Virus (YFV) or dengue serotype 4 was defined as the outgroup (data not shown). For these reasons, ML tree rooted with molecular clock was used as the template for simulating ancestral sequence. A rooted ML tree of 189 dengue sequences is shown in FIG. 3.

The simulation of ancestral sequences at each internal node was done with “baseml” program in PAML package for both marginal and joint ancestral reconstruction (42). The tree shown in FIG. 3 served as the template. We reconstruct ancestral sequences at nucleotide level rather than at codon or amino acid levels since the later two approaches ignore synonymous substitutions that may also experience positive selection (25). An ancestral sequence at the deepest root of the tree was inferred successfully (FIG. 3). This sequence contains a stop codon (TAA) at amino acid position 227, originating from the ancestral sequence of dengue serotypes 1 and 3 due to a replacement of cytosine by adenine at both nucleotide positions 680 and 681. We then examined the posterior probability of the reconstruction at these two positions. There is a lower posterior probability at nucleotide 680 comparing to 681. Adenine at nucleotide 680 is therefore replaced by cytosine, resulting in an amino acid serine at position 227, which is consistent with the ancestral reconstruction at the amino acid level (data not shown). A final ancestral envelope sequence (1485 bp) of all dengue serotypes, named DengueA1, is shown as SEQ ID NO:1.

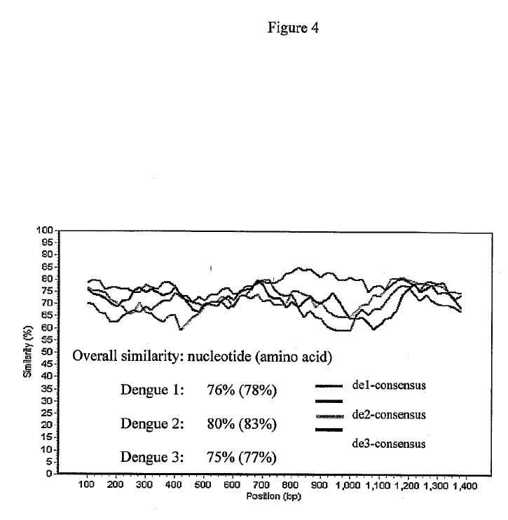

The similarity of DengueA1 was examined at both nucleotide and amino acid levels using SimPlot program (23). DengueA1 shows 77% nucleotide homogeneity against each consensus dengue serotype (FIG. 4). Since average similarity at the nucleotide level is approximately 66.7% among wild-type dengue serotypes, an enhancement of similarity of 10.3% is achieved by DengueA1.

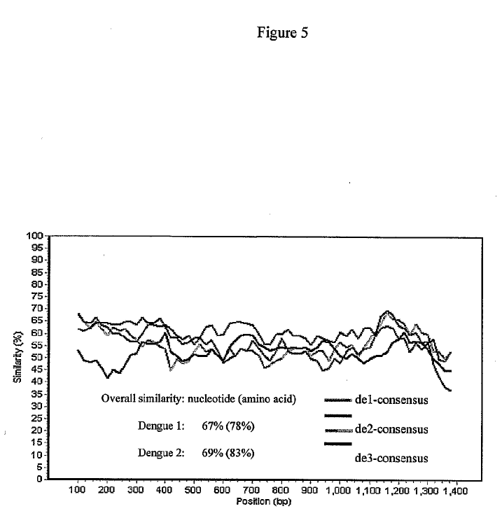

Most of amino acids are encoded by more than one codon. For a given amino acid, different species may favor different codons, which creates possible codon bias. There is documented effect of codon bias on the expression of viral genes (1). We examined the codon usage for ancestral envelope gene (DengueA1) and wild-type dengue envelope gene (189 isolates) and found an obvious difference of codon usage between dengue virus and mammal species (Table 4), a situation very similar to HIV (1). The codon usage of DengueA1 was then optimized based on mammal species. The processing of optimization was done with program JCat (13) that implanted an algorithm for the calculation of the codon adaption index (CAI). The CAI is the prevailing empirical measure of expressivity (28). The nucleotide sequence of codon-optimized DengueA1 is shown in the sequence listing as SEQ ID NO:2 and named as DengueA2. DengueA1 and DengueA2 share 71% homogeneity at the nucleotide acid level although they encode the same amino acid sequence. DengueA2 shows 67% nucleotide homogeneity against each wild-type consensus dengue serotype, a 10% drop comparing to DengueA1 (FIG. 5).

TABLE 4
Codon usages of ancestral envelope gene, wild-type
dengue envelope gene and mammal species.

| | Codon | DengueA1 | Wild | Mamm |
|---|---|---|---|---|
| aa | | | | |
| Ala | GCU | 23.5 | 26.4 | 28.8 |
| | GCC | 14.7 | 26.4 | 40.2 |
| | GCA | 52.9 | 34.7 | 21.0 |
| | GCG | 8.8 | 12.6 | 9.9 |
| Arg | CGU | 0 | 6.3 | 9.1 |
| | CGC | 0 | 9.4 | 19.5 |
| | CGA | 13.3 | 8.8 | 10.7 |
| | CGG | 0 | 2.5 | 18.2 |
| | AGA | 80 | 50.3 | 21.0 |
| | AGG | 6.7 | 22.6 | 21.5 |
| Asn | AAU | 61.1 | 47.0 | 41.4 |
| | AAC | 38.9 | 53.0 | 58.6 |
| Asp | GAU | 44.4 | 34.8 | 42.6 |
| | GAC | 55.6 | 65.2 | 57.4 |
| Cys | UGU | 53.8 | 46.0 | 42.7 |
| | UGC | 46.2 | 54.0 | 57.3 |
| Gln | CAA | 64.3 | 56.5 | 26.1 |
| | CAG | 35.7 | 43.5 | 73.9 |
| Glu | GAA | 77.4 | 66.0 | 39.9 |
| | GAG | 22.6 | 34.0 | 60.1 |
| Aa | | | | |
| Gly | GGU | 11.8 | 10.3 | 17.6 |
| | GGC | 2.1 | 11.3 | 34.1 |
| | GGA | 76.5 | 60.6 | 25.5 |
| | GGG | 9.8 | 17.9 | 22.8 |
| His | CAU | 72.7 | 53.0 | 38.8 |
| | CAC | 27.3 | 47.0 | 61.2 |
| Ile | AUU | 34.5 | 27.0 | 33.3 |
| | AUC | 17.2 | 28.4 | 53.6 |
| | AUA | 48.3 | 44.6 | 13.1 |
| Leu | UUA | 17.1 | 13.7 | 5.4 |
| | UUG | 25.8 | 22.5 | 12.2 |
| | CUU | 17.1 | 5.6 | 12.1 |
| | CUC | 2.9 | 12.0 | 20.8 |
| | CUA | 17.1 | 17.9 | 6.8 |
| | CUG | 20 | 28.2 | 42.8 |
| Lys | AAA | 68.2 | 59.7 | 37.6 |
| | AAG | 31.8 | 40.3 | 62.4 |
| Phe | UUU | 84.2 | 58.8 | 40.7 |
| | UUC | 15.8 | 41.2 | 59.3 |
| aa | | | | |
| Pro | CCU | 43.8 | 31.9 | 28.8 |
| | CCC | 6.2 | 18.7 | 32.7 |
| | CCA | 43.8 | 41.0 | 27.3 |
| | CCG | 6.2 | 8.4 | 11.2 |
| Ser | UCU | 18.2 | 14.1 | 18.3 |
| | UCC | 0 | 12.7 | 23.5 |
| | UCA | 54.6 | 39.5 | 14.2 |
| | UCG | 0 | 6.9 | 5.9 |
| | AGU | 13.6 | 9.6 | 13.4 |
| | AGC | 13.6 | 17.2 | 24.8 |
| Thr | ACU | 15.2 | 11.7 | 23.4 |
| | ACC | 0 | 19.5 | 38.5 |
| | ACA | 80.4 | 54.7 | 26.4 |
| | ACG | 4.4 | 14.1 | 11.8 |
| Tyr | UAU | 57.1 | 46.0 | 40.2 |
| | UAC | 42.9 | 54.0 | 59.8 |
| Val | GUU | 36.6 | 20.9 | 16.4 |
| | GUC | 7.3 | 24.8 | 25.6 |
| | GUA | 22 | 16.2 | 9.9 |
| | GUG | 34.1 | 38.1 | 48.1 |

The codon usage of mammal species is from the work of Cherry (5). Program MEGA was used to generate the codon usage of dengue viruses. Mamm, mammal species; Wild, wild-type dengue virus isolates (n = 189).

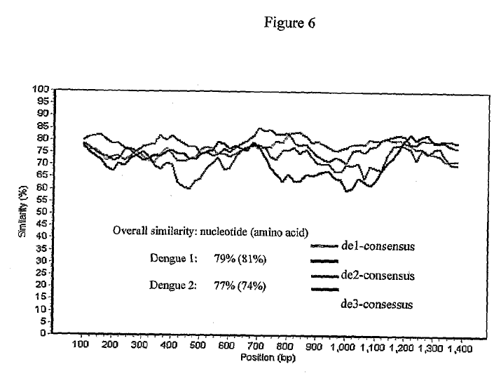

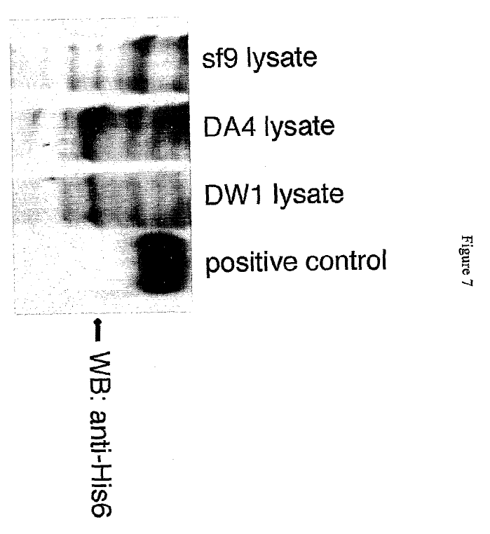

The consensus sequence is defined as a sequence in which nucleic acids at each position have the highest frequency within a given sequence data set. Based on this principle, we produced consensus sequences for each wild-type dengue serotype with the assistance of multiple programs implanted in Wisconsin GCG package (39). A consensus sequence for all four dengue serotypes was also produced and named as DengueC. To avoid numerical bias, we included only 30 isolates for each dengue virus serotype by excluding homogeneous isolates. DengueC was finally derived from 120 dengue virus isolates. As expected, DengueC has 78.5% nucleotide homogeneity to each wild-type consensus dengue serotype (FIG. 6), similar to DengueA1 (77%). The nucleotide sequence of DengueC is shown in the sequence listing as SEQ ID NO:3.

The assembly strategy is similar to that has been described by inventor for hepatitis C virus (6). Briefly, the assembly process consists of multiple rounds of PCR—gel purification—PCR. The plasmid containing wild-type dengue-1 envelope sequence, kindly provided by Dr.

Robert Putnak in Walter Reed Army Institute of Research (WRAIR), was used as the initial template for PCR assembly. Mismatched sequences were corrected by 5′ end primer extension. To reduce possible mutations induced by Taq DNA polymerase, the number of cycles for each PCR round was decreased to 20. Each PCR product was gel-purified and served as the template for the next PCR round. The final product of PCR-assembly was ligated into pUC19 vector for the production of recombinant clones. Correct clones were identified by full sequencing.

### Example 2

### Expression of Ancestral Dengue Envelope Gene

Synthesis of Ancestral Dengue Envelope Gene, Dengue A2.

Dengue A2 is a codon-optimized ancestral envelope gene of all four dengue serotypes through evolutionary simulation. We applied the PCR-based assembly strategy for the synthesis of Dengue A2. In doing so, Dengue A2 was divided into three domains (D1, D2 and D3) and each domain was first synthesized individually (FIGS. 9-11). The final assembly reaction with fragments D1, D2 and D3 generated Dengue A2, which was cloned into pUC19 vector. Ten recombinant clones were fully sequenced. The clone DA4 was selected for further correction of mismatched nucleotide sites by using site-directed mutagenesis kit (Stratagene).

Plasmid Construction.

The clone DA4 was fused with a six his tag sequence at the 3′ end and a signal sequence (105 bp) at the 5′ end, which was derived from wild-type dengue 1 serotype (Genbank accession number U88535). The clone DA4 was then subcloned to plasmid pBacPAK9 vector (shuttle vector for baculovirus expression system from ClonTech) and the correct insert was confirmed by fully sequencing. As a control, full-length wild-type dengue envelope gene was amplified from plasmid pVAXcd11 that contains wild-type dengue serotype 1 prim and E genes (Genbank accession number U88535), a gift from Dr. Robert Putnam in Walter Reed Army Institute of Research. The PCR product was processed as the clone DA4 and a correct clone, DW1, was identified. Thus DA4 and DW1 have the same expression cassette except different dengue E genes, a synthesized ancestor for DA4 and a wild-type dengue envelope gene for DW1.

Expression in Insect Cells.

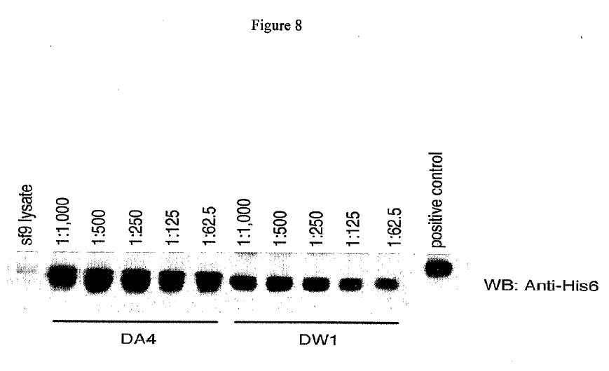

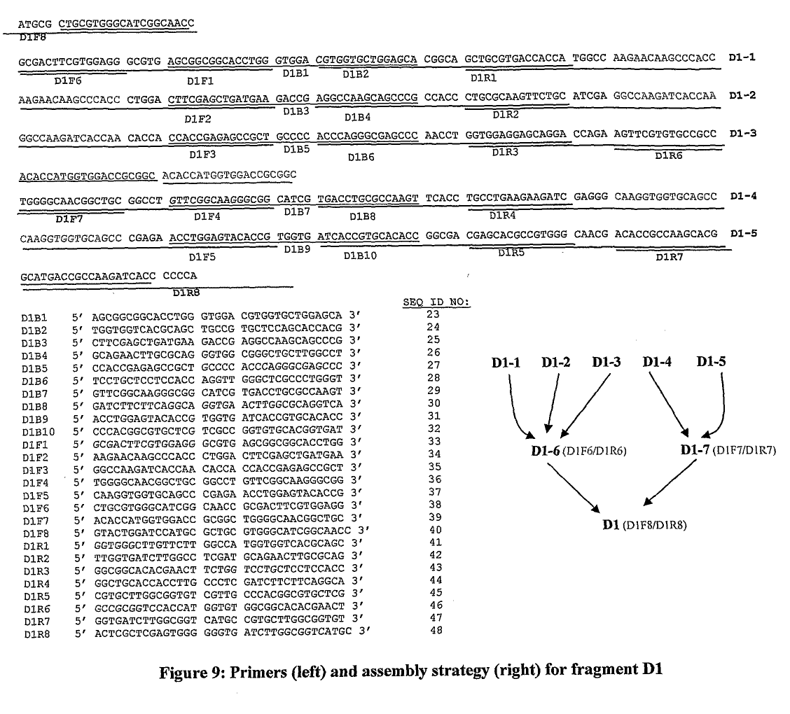

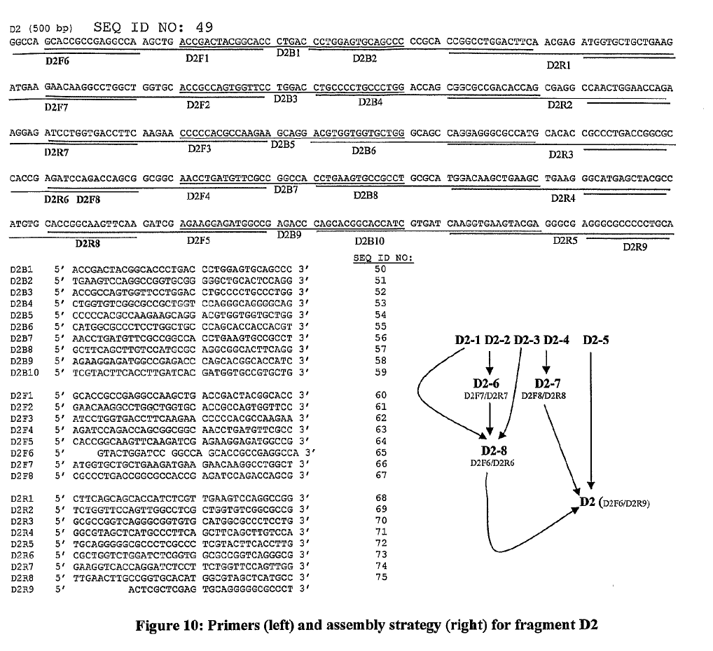

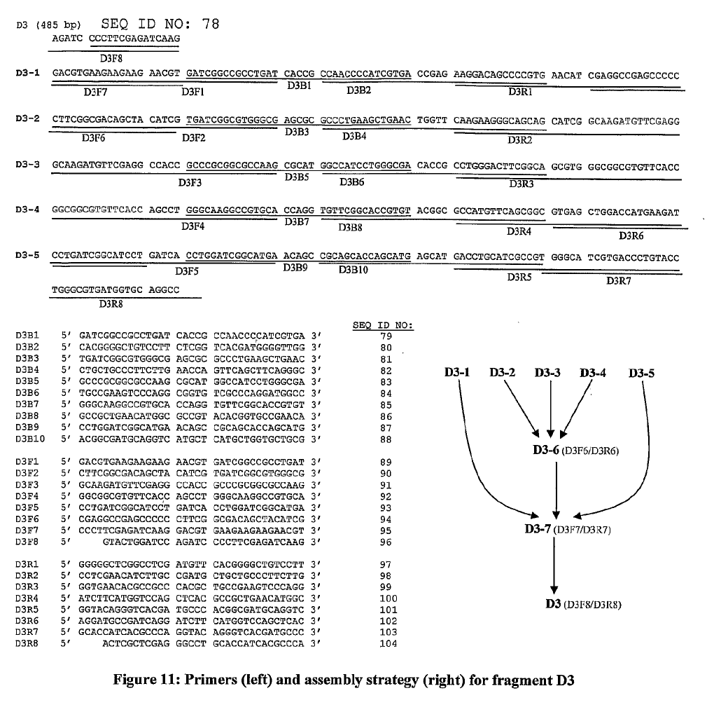

Protein expression was done with Backpack Baculovirus Expression System (Clontech) following the instruction. As expected, a ˜55 kd protein was detected by immune blotting and clone DA4 showed the higher yield than clone DW1, indicating the codon optimization did play a role (FIGS. 7 and 8).

Protein Purification.

We tried two different approaches for protein purification, the immobilized metal affinity chromatography (IMAC) by using his tag and 30% sucrose ultracentrifugation (see references 43, 44).

1-36. (canceled)

37. A composition comprising a polynucleotide that comprises a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:21.

38. The composition of claim 1 wherein said polynucleotide comprises a sequence as set forth in SEQ ID NO:1.

39. The composition of claim 2 wherein said sequence is at least 81% identical to SEQ ID NO:1.

40. The composition of claim 1 wherein said polynucleotide comprises a sequence as set forth in SEQ ID NO:2.

41. The composition of claim 4 wherein said sequence is at least 88% identical to SEQ ID NO:2.

42. The composition of claim 1 wherein said polynucleotide comprises a sequence as set forth in SEQ ID NO:3.

43. The composition of claim 6 wherein said sequence is at least 81% identical to SEQ ID NO:3.

44. The composition of claim 1 wherein said polynucleotide comprises a sequence as set forth in SEQ ID NO:12.

45. The composition of claim 8 wherein said sequence is at least 84% identical to SEQ ID NO:12.

46. The composition of claim 1 wherein said polynucleotide comprises a sequence as set forth in SEQ ID NO:13.

47. The composition of claim 10 wherein said sequence is at least 82% identical to SEQ ID NO:13.

48. A composition comprising a polynucleotide encoding a polypeptide having a sequence selected from the group consisting of SEQ ID NO:12 and SEQ ID NO:13.

49. The composition of claim 12 wherein said sequence is at least 88% identical to SEQ ID NO:12.

50. The composition of claim 12 wherein said sequence is at least 84% identical to SEQ ID NO:12.

51. The composition of claim 12 wherein said sequence is at least 88% identical to SEQ ID NO:13.

52. The composition of claim 12 wherein said sequence is at least 82% identical to SEQ ID NO:13.

53. An isolated polynucleotide comprising a sequence as set forth in SEQ ID NO:2.

54. A polypeptide comprising a sequence selected from the group consisting of SEQ ID NO:12 and SEQ ID NO:12.


SEQUENCE LISTING (103 sequences)

SEQ ID NO: 1 - DNA, 1485 bp, Artificial
  computer - conceptual generated
atgcgatgtgtaggaataggaaacagagactttgtggaaggagtttcaggaggaacatgggttgatgtggtgctagaacatggaagttgtgttacaactatggcaaagaataaaccaacattggactttgaactgatgaagacagaagccaaacaaccggctactttaaggaaattttgtattgaagctaaaataacaaacacaacaacagaatcaagatgtccaacacaaggagaacctaatctagtagaggaacaagaccaaaagttcgtttgcagacatacgatggtagacagaggttggggtaatggttgtggtttgtttggaaaaggaggcattgtgacatgtgcgaagtttacatgcttgaagaaaatagaaggaaaagtggtgcaacctgaaaacttagaatacacagtggtcataacagttcacacaggagatgaacacgcagtgggaaatgatacagcaaaacatggaatgacagccaagataacaccccaggcatcaactgcagaagctaaactgacagactatggaactcttacactggaatgttcaccaagaacaggtcttgattttaatgagatggttttgttgaaaatgaaaaataaagcatggcttgtgcatagacaatggttcttagacttacctctaccatggacatcaggagctgacacatcagaagcaaattggaatcagaaagagatactggtgacattcaaaaatcctcatgcaaagaaacaggatgtagttgttctaggatctcaagaaggagccatgcatacagcacttacaggagctacagaaatccaaacgtcaggaggaaacttaatgtttgcaggacatctaaagtgcagactgagaatggataaactgaaactcaaagggatgtcatacgcgatgtgcacaggaaagtttaaaattgagaaagaaatggcagaaacacagcatggaacaatagttattaaagttaagtatgaaggagaaggtgcaccatgcaagatcccttttgagataaaggatgtgaaaaagaaaaatgttattgggcgattgattacagctaacccaatagtaacagagaaagacagtccagtcaacattgaagcagaacctccttttggggacagctacatcgtgataggagtaggagaaagagcattgaaacttaactggtttaagaaaggaagctctattgggaaaatgtttgaggcaacagccagaggagcaaaaagaatggccattttgggagacacagcttgggattttggatctgtaggaggagtgtttacatcattaggaaaagcggtacaccaggtttttggaactgtctatggagctatgtttagtggagtttcatggactatgaaaatcctaatagggattcttataacatggataggaatgaattcaagaagcacttctatgtcaatgacatgcatagcagtaggaatcgttacactgtatttgggagttatggtgcaagca

SEQ ID NO: 2 - DNA, 1485 bp, Artificial
  computer - conceptual generated
atgcgctgcgtgggcatcggcaaccgcgacttcgtggagggcgtgagcggcggcacctgggtggacgtggtgctggagcacggcagctgcgtgaccaccatggccaagaacaagcccaccctggacttcgagctgatgaagaccgaggccaagcagcccgccaccctgcgcaagttctgcatcgaggccaagatcaccaacaccaccaccgagagccgctgccccacccagggcgagcccaacctggtggaggagcaggaccagaagttcgtgtgccgccacaccatggtggaccgcggctggggcaacggctgcggcctgttcggcaagggcggcatcgtgacctgcgccaagttcacctgcctgaagaagatcgagggcaaggtggtgcagcccgagaacctggagtacaccgtggtgatcaccgtgcacaccggcgacgagcacgccgtgggcaacgacaccgccaagcacggcatgaccgccaagatcaccccccaggccagcaccgccgaggccaagctgaccgactacggcaccctgaccctggagtgcagcccccgcaccggcctggacttcaacgagatggtgctgctgaagatgaagaacaaggcctggctggtgcaccgccagtggttcctggacctgcccctgccctggaccagcggcgccgacaccagcgaggccaactggaaccagaaggagatcctggtgaccttcaagaacccccacgccaagaagcaggacgtggtggtgctgggcagccaggagggcgccatgcacaccgccctgaccggcgccaccgagatccagaccagcggcggcaacctgatgttcgccggccacctgaagtgccgcctgcgcatggacaagctgaagctgaagggcatgagctacgccatgtgcaccggcaagttcaagatcgagaaggagatggccgagacccagcacggcaccatcgtgatcaaggtgaagtacgagggcgagggcgccccctgcaagatccccttcgagatcaaggacgtgaagaagaagaacgtgatcggccgcctgatcaccgccaaccccatcgtgaccgagaaggacagccccgtgaacatcgaggccgagccccccttcggcgacagctacatcgtgatcggcgtgggcgagcgcgccctgaagctgaactggttcaagaagggcagcagcatcggcaagatgttcgaggccaccgcccgcggcgccaagcgcatggccatcctgggcgacaccgcctgggacttcggcagcgtgggcggcgtgttcaccagcctgggcaaggccgtgcaccaggtgttcggcaccgtgtacggcgccatgttcagcggcgtgagctggaccatgaagatcctgatcggcatcctgatcacctggatcggcatgaacagccgcagcaccagcatgagcatgacctgcatcgccgtgggcatcgtgaccctgtacctgggcgtgatggtgcaggcc

SEQ ID NO: 3 - DNA, 1485 bp, Artificial
  computer - conceptual generated
atgcgatgcgtgggagtaggaaacagagactttgtggaaggagtgtcaggaggaacgtgggttgacgtggtgctagaacatggaggctgtgtgacaaccatggcaaagaacaaaccaacattggattttgaactgatgaagacagaagccacgcaaccggccaccctaaggaagttttgcattgaagcaaaaatatccaacacaacaaccgaatcaagatgtccaacacaaggggaacccactctgaatgaagaacaggaccagaagtacgtgtgcaggcgaaccttggtagacagaggctggggaaatggctgtggattgtttggaaaaggaagctttgtgacatgtgcgaagtttaaatgtttggaaaaaatagaaggaaaagtggtgcaacatgaaaacctgaaatacacagtgatcgtaacagtccacacaggagaccagcaccaagtgggaaatgacacagcaaaacatggaatgacagccaagataacaccccaggcaccaacggcagaagtaaaattgccagactatggagcccttacactggattgctcaccaagaacaggactggacttcaatgagatggtgttgttgaaaatgaaaaacaaagcatggctggtgcacaagcaatggtttttagacctacctctaccatggacatcaggagctgcaacatcagaagcaaattggaacaagaaagagatactggtgacattcaaaaatcctcatgcaaagaaacaggaagtagttgtcctaggatctcaagaaggagcaatgcacacagcactgacaggagccacagaaatccaaacgtcaggaggaacaaaaatttttgcaggacacctgaaatgcagacttaaaatggacaaactgaaactcaaggggatgtcatatgcgatgtgcacaggaacgtttaagtttgagaaagaagtggcagaaacacagcatggaacaatagtcgtgaaggttaagtatgaaggggcagatgctccatgcaagatccctttttcgataaaagatgtgaaagggaaaaatcagaatgggagactgatcacagccaacccaatggttactgagaaagacagaccagtcaacattgaagcagaacctccttttggggacagctacatagtgataggagtaggagacagagcattgaaactcaactggttcaagaaaggaagctccattgggaagatgtttgaggccacagccagaggagcaaagagaatggccattttgggagacacagcctgggactttggttctgtgggaggagtgttcacatcattaggaaaagcggtacaccaggtttttggaagtgtgtatacagctctgtttagtggagtctcatggatgatgaaaatcgtaataggggtcctcttgacatggataggaatgaattcaagaaacacttcaatgtcaatgtcatgcatagcggttggaatcatcacactgtatctgggagtcatggttcaagct

SEQ ID NO: 4 - DNA, 66 bp, Artificial
  computer - conceptual generated
atgcgatgtgtaggaataggaaacagagactttgtggaaggagtttcaggaggaacatgggttgat

SEQ ID NO: 5 - DNA, 78 bp, Artificial
  computer - conceptual generated
aaattttgtattgaagctaaaataacaaacacaacaacagaatcaagatgtccaacacaaggagaacctaatctagta

SEQ ID NO: 6 - DNA, 51 bp, Artificial
  computer - conceptual generated
aatggttgtggtttgtttggaaaaggaggcattgtgacatgtgcgaagttt

SEQ ID NO: 7 - DNA, 391 bp, Artificial
  computer - conceptual generated
gcagtgggaaatgatacagcaaaacatggaatgacagccaagataacaccccaggcatcaactgcagaagctaaactgacagactatggaactcttacactggaatgttcaccaagaacaggtcttgattttaatgagatggttttgttgaaaatgaaaaataaagcatggcttgtgcatagacaatggttcttagacttacctctaccatggacatcaggagctgacacatcagaagcaaattggaatcagaaagagatactggtgacattcaaaaatcctcatgcaaagaaacaggatgtagttgttctaggatctcaagaaggagccatgcatacagcacttacaggagctacagaaatccaaacgtcaggaggaaacttaatgtttg

SEQ ID NO: 8 - DNA, 139 bp, Artificial
  computer - conceptual generated
acagcttgggattttggatctgtaggaggagtgtttacatcattaggaaaagcggtacaccaggtttttggaactgtctatggagctatgtttagtggagtttcatggactatgaaaatcctaatagggattcttataa

SEQ ID NO: 9 - DNA, 25 bp, Artificial
  computer - conceptual generated
atgcgatgtgtaggaataggaaaca

SEQ ID NO: 10 - DNA, 20 bp, Artificial
  computer - conceptual generated
tcaggagctgacacatcaga

SEQ ID NO: 11 - DNA, 30 bp, Artificial
  computer - conceptual generated
gtgtttacatcattaggaaaagcggtacac

SEQ ID NO: 12 - protein, 495 aa, Artificial
  computer - conceptual generated
MRCVGIGNRDFVEGVSGGTWVDVVLEHGSCVTTMAKNKPTLDFELMKTEAKQPATLRKFCIEAKITNTTTESRCPTQGEPNLVEEQDQKFVCRHTMVDRGWGNGCGLFGKGGIVTCAKFTCLKKIEGKVVQPENLEYTVVITVHTGDEHAVGNDTAKHGMTAKITPQASTAEAKLTDYGTLTLECSPRTGLDFNEMVLLKMKNKAWLVHRQWFLDLPLPWTSGADTSEANWNQKEILVTFKNPHAKKQDVVVLGSQEGAMHTALTGATEIQTSGGNLMFAGHLKCRLRMDKLKLKGMSYAMCTGKFKIEKEMAETQHGTIVIKVKYEGEGAPCKIPFEIKDVKKKNVIGRLITANPIVTEKDSPVNIEAEPPFGDSYIVIGVGERALKLNWFKKGSSIGKMFEATARGAKRMAILGDTAWDFGSVGGVFTSLGKAVHQVFGTVYGAMFSGVSWTMKILIGILITWIGMNSRSTSMSMTCIAVGIVTLYLGVMVQA

SEQ ID NO: 13 - protein, 495 aa, Artificial
  computer - conceptual generated
MRCVGVGNRDFVEGVSGGTWVDVVLEHGGCVTTMAKNKPTLDFELMKTEATQPATLRKFCIEAKISNTTTESRCPTQGEPTLNEEQDQKYVCRRTLVDRGWGNGCGLFGKGSFVTCAKFKCLEKIEGKVVQHENLKYTVIVTVHTGDQHQVGNDTAKHGMTAKITPQAPTAEVKLPDYGALTLDCSPRTGLDFNEMVLLKMKNKAWLVHKQWFLDLPLPWTSGAATSEANWNKKEILVTFKNPHAKKQEVVVLGSQEGAMHTALTGATEIQTSGGTKIFAGHLKCRLKMDKLKLKGMSYAMCTGTFKFEKEVAETQHGTIVVKVKYEGADAPCKIPFSIKDVKGKNQNGRLITANPMVTEKDRPVNIEAEPPFGDSYIVIGVGDRALKLNWFKKGSSIGKMFEATARGAKRMAILGDTAWDFGSVGGVFTSLGKAVHQVFGSVYTALFSGVSWMMKIVIGVLLTWIGMNSRNTSMSMSCIAVGIITLYLGVMVQA

SEQ ID NO: 14 - protein, 22 aa, Artificial
  computer - conceptual generated
MRCVGIGNRDFVEGVSGGTWVD

SEQ ID NO: 15 - protein, 25 aa, Artificial
  computer - conceptual generated
FCIEAKITNTTTESRCPTQGEPNLV

SEQ ID NO: 16 - protein, 19 aa, Artificial
  computer - conceptual generated
WGNGCGLFGKGGIVTCAKF

SEQ ID NO: 17 - protein, 131 aa, Artificial
  computer - conceptual generated
HAVGNDTAKHGMTAKITPQASTAEAKLTDYGTLTLECSPRTGLDFNEMVLLKMKNKAWLVHRQWFLDLPLPWTSGADTSEANWNQKEILVTFKNPHAKKQDVVVLGSQEGAMHTALTGATEIQTSGGNLMF

SEQ ID NO: 18 - protein, 53 aa, Artificial
  computer - conceptual generated
RMAILGDTAWDFGSVGGVFTSLGKAVHQVFGTVYGAMFSGVSWTMKILIGILI

SEQ ID NO: 19 - protein, 8 aa, Artificial
  computer - conceptual generated
MRCVGIGN

SEQ ID NO: 20 - protein, 8 aa, Artificial
  computer - conceptual generated
TSGADTSE

SEQ ID NO: 21 - protein, 11 aa, Artificial
  computer - conceptual generated
GVFTSLGKAVH

SEQ ID NO: 22 - DNA, 565 bp, Artificial
  synthetic fragment
atgcgctgcgtgggcatcggcaaccgcgacttcgtggagggcgtgagcggcggcacctgggtggacgtggtgctggagcacggcagctgcgtgaccaccatggccaagaacaagcccaccaagaacaagcccaccctggacttcgagctgatgaagaccgaggccaagcagcccgccaccctgcgcaagttctgcatcgaggccaagatcaccaaggccaagatcaccaacaccaccaccgagagccgctgccccacccagggcgagcccaacctggtggaggagcaggaccagaagttcgtgtgccgccacaccatggtggaccgcggcacaccatggtggaccgcggctggggcaacggctgcggcctgttcggcaagggcggcatcgtgacctgcgccaagttcacctgcctgaagaagatcgagggcaaggtggtgcagcccaaggtggtgcagcccgagaacctggagtacaccgtggtgatcaccgtgcacaccggcgacgagcacgccgtgggcaacgacaccgccaagcacggcatgaccgccaagatcacccccca

SEQ ID NO: 23 - DNA, 35 bp, Artificial
  synthetic
agcggcggcacctgggtggacgtggtgctggagca

SEQ ID NO: 24 - DNA, 35 bp, Artificial
  synthetic
tggtggtcacgcagctgccgtgctccagcaccacg

SEQ ID NO: 25 - DNA, 35 bp, Artificial
  synthetic
cttcgagctgatgaagaccgaggccaagcagcccg

SEQ ID NO: 26 - DNA, 35 bp, Artificial
  synthetic
gcagaacttgcgcagggtggcgggctgcttggcct

SEQ ID NO: 27 - DNA, 35 bp, Artificial
  synthetic
ccaccgagagccgctgccccacccagggcgagccc

SEQ ID NO: 28 - DNA, 35 bp, Artificial
  synthetic
tcctgctcctccaccaggttgggctcgccctgggt

SEQ ID NO: 29 - DNA, 35 bp, Artificial
  synthetic
gttcggcaagggcggcatcgtgacctgcgccaagt

SEQ ID NO: 30 - DNA, 35 bp, Artificial
  synthetic
gatcttcttcaggcaggtgaacttggcgcaggtca

SEQ ID NO: 31 - DNA, 35 bp, Artificial
  synthetic
acctggagtacaccgtggtgatcaccgtgcacacc

SEQ ID NO: 32 - DNA, 35 bp, Artificial
  synthetic
cccacggcgtgctcgtcgccggtgtgcacggtgat

SEQ ID NO: 33 - DNA, 35 bp, Artificial
  synthetic
gcgacttcgtggagggcgtgagcggcggcacctgg

SEQ ID NO: 34 - DNA, 35 bp, Artificial
  synthetic
aagaacaagcccaccctggacttcgagctgatgaa

SEQ ID NO: 35 - DNA, 35 bp, Artificial
  synthetic
ggccaagatcaccaacaccaccaccgagagccgct

SEQ ID NO: 36 - DNA, 35 bp, Artificial
  synthetic
tggggcaacggctgcggcctgttcggcaagggcgg

SEQ ID NO: 37 - DNA, 35 bp, Artificial
  synthetic
caaggtggtgcagcccgagaacctggagtacaccg

SEQ ID NO: 38 - DNA, 35 bp, Artificial
  synthetic
ctgcgtgggcatcggcaaccgcgacttcgtggagg

SEQ ID NO: 39 - DNA, 35 bp, Artificial
  synthetic
acaccatggtggaccgcggctggggcaacggctgc

SEQ ID NO: 40 - DNA, 36 bp, Artificial
  synthetic
gtactggatccatgcgctgcgtgggcatcggcaacc

SEQ ID NO: 41 - DNA, 35 bp, Artificial
  synthetic
ggtgggcttgttcttggccatggtggtcacgcagc

SEQ ID NO: 42 - DNA, 35 bp, Artificial
  synthetic
ttggtgatcttggcctcgatgcagaacttgcgcag

SEQ ID NO: 43 - DNA, 35 bp, Artificial
  synthetic
ggcggcacacgaacttctggtcctgctcctccacc

SEQ ID NO: 44 - DNA, 35 bp, Artificial
  synthetic
ggctgcaccaccttgccctcgatcttcttcaggca

SEQ ID NO: 45 - DNA, 35 bp, Artificial
  synthetic
cgtgcttggcggtgtcgttgcccacggcgtgctcg

SEQ ID NO: 46 - DNA, 35 bp, Artificial
  synthetic
gccgcggtccaccatggtgtggcggcacacgaact

SEQ ID NO: 47 - DNA, 35 bp, Artificial
  synthetic
ggtgatcttggcggtcatgccgtgcttggcggtgt

SEQ ID NO: 48 - DNA, 36 bp, Artificial
  synthetic
actcgctcgagtggggggtgatcttggcggtcatgc

SEQ ID NO: 49 - DNA, 500 bp, Artificial
  synthetic
ggccagcaccgccgaggccaagctgaccgactacggcaccctgaccctggagtgcagcccccgcaccggcctggacttcaacgagatggtgctgctgaagatgaagaacaaggcctggctggtgcaccgccagtggttcctggacctgcccctgccctggaccagcggcgccgacaccagcgaggccaactggaaccagaaggagatcctggtgaccttcaagaacccccacgccaagaagcaggacgtggtggtgctgggcagccaggagggcgccatgcacaccgccctgaccggcgccaccgagatccagaccagcggcggcaacctgatgttcgccggccacctgaagtgccgcctgcgcatggacaagctgaagctgaagggcatgagctacgccatgtgcaccggcaagttcaagatcgagaaggagatggccgagacccagcacggcaccatcgtgatcaaggtgaagtacgagggcgagggcgccccctgca

SEQ ID NO: 50 - DNA, 35 bp, Artificial
  synthetic
accgactacggcaccctgaccctggagtgcagccc

SEQ ID NO: 51 - DNA, 35 bp, Artificial
  synthetic
tgaagtccaggccggtgcgggggctgcactccagg

SEQ ID NO: 52 - DNA, 35 bp, Artificial
  synthetic
accgccagtggttcctggacctgcccctgccctgg

SEQ ID NO: 53 - DNA, 35 bp, Artificial
  synthetic
ctggtgtcggcgccgctggtccagggcaggggcag

SEQ ID NO: 54 - DNA, 35 bp, Artificial
  synthetic
cccccacgccaagaagcaggacgtggtggtgctgg

SEQ ID NO: 55 - DNA, 35 bp, Artificial
  synthetic
catggcgccctcctggctgcccagcaccaccacgt

SEQ ID NO: 56 - DNA, 35 bp, Artificial
  synthetic
aacctgatgttcgccggccacctgaagtgccgcct

SEQ ID NO: 57 - DNA, 35 bp, Artificial
  synthetic
gcttcagcttgtccatgcgcaggcggcacttcagg

SEQ ID NO: 58 - DNA, 35 bp, Artificial
  synthetic
agaaggagatggccgagacccagcacggcaccatc

SEQ ID NO: 59 - DNA, 35 bp, Artificial
  synthetic
tcgtacttcaccttgatcacgatggtgccgtgctg

SEQ ID NO: 60 - DNA, 35 bp, Artificial
  synthetic
gcaccgccgaggccaagctgaccgactacggcacc

SEQ ID NO: 61 - DNA, 35 bp, Artificial
  synthetic
gaacaaggcctggctggtgcaccgccagtggttcc

SEQ ID NO: 62 - DNA, 35 bp, Artificial
  synthetic
atcctggtgaccttcaagaacccccacgccaagaa

SEQ ID NO: 63 - DNA, 35 bp, Artificial
  synthetic
agatccagaccagcggcggcaacctgatgttcgcc

SEQ ID NO: 64 - DNA, 35 bp, Artificial
  synthetic
caccggcaagttcaagatcgagaaggagatggccg

SEQ ID NO: 65 - DNA, 31 bp, Artificial
  synthetic
gtactggatccggccagcaccgccgaggcca

SEQ ID NO: 66 - DNA, 35 bp, Artificial
  synthetic
atggtgctgctgaagatgaagaacaaggcctggct

SEQ ID NO: 67 - DNA, 35 bp, Artificial
  synthetic
cgccctgaccggcgccaccgagatccagaccagcg

SEQ ID NO: 68 - DNA, 35 bp, Artificial
  synthetic
cttcagcagcaccatctcgttgaagtccaggccgg

SEQ ID NO: 69 - DNA, 35 bp, Artificial
  synthetic
tctggttccagttggcctcgctggtgtcggcgccg

SEQ ID NO: 70 - DNA, 35 bp, Artificial
  synthetic
gcgccggtcagggcggtgtgcatggcgccctcctg

SEQ ID NO: 71 - DNA, 35 bp, Artificial
  synthetic
ggcgtagctcatgcccttcagcttcagcttgtcca

SEQ ID NO: 72 - DNA, 35 bp, Artificial
  synthetic
tgcagggggcgccctcgccctcgtacttcaccttg

SEQ ID NO: 73 - DNA, 35 bp, Artificial
  synthetic
cgctggtctggatctcggtggcgccggtcagggcg

SEQ ID NO: 74 - DNA, 35 bp, Artificial
  synthetic
gaaggtcaccaggatctccttctggttccagttgg

SEQ ID NO: 75 - DNA, 35 bp, Artificial
  synthetic
ttgaacttgccggtgcacatggcgtagctcatgcc

SEQ ID NO: 76 - DNA, 26 bp, Artificial
  synthetic
actcgctcgagtgcagggggcgccct

SEQ ID NO: 77 - DNA, 515 bp, Artificial
  synthetic
agatccccttcgagatcaaggacgtgaagaagaagaacgtgatcggccgcctgatcaccgccaaccccatcgtgaccgagaaggacagccccgtgaacatcgaggccgagccccccttcggcgacagctacatcgtgatcggcgtgggcgagcgcgccctgaagctgaactggttcaagaagggcagcagcatcggcaagatgttcgagggcaagatgttcgaggccaccgcccgcggcgccaagcgcatggccatcctgggcgacaccgcctgggacttcggcagcgtgggcggcgtgttcaccggcggcgtgttcaccagcctgggcaaggccgtgcaccaggtgttcggcaccgtgtacggcgccatgttcagcggcgtgagctggaccatgaagatcctgatcggcatcctgatcacctggatcggcatgaacagccgcagcaccagcatgagcatgacctgcatcgccgtgggcatcgtgaccctgtacctgggcgtgatggtgcaggcc

SEQ ID NO: 78 - DNA, 35 bp, Artificial
  synthetic
gatcggccgcctgatcaccgccaaccccatcgtga

SEQ ID NO: 79 - DNA, 35 bp, Artificial
  synthetic
cacggggctgtccttctcggtcacgatggggttgg

SEQ ID NO: 80 - DNA, 35 bp, Artificial
  synthetic
tgatcggcgtgggcgagcgcgccctgaagctgaac

SEQ ID NO: 81 - DNA, 35 bp, Artificial
  synthetic
ctgctgcccttcttgaaccagttcagcttcagggc

SEQ ID NO: 82 - DNA, 35 bp, Artificial
  synthetic
gcccgcggcgccaagcgcatggccatcctgggcga

SEQ ID NO: 83 - DNA, 35 bp, Artificial
  synthetic
tgccgaagtcccaggcggtgtcgcccaggatggcc

SEQ ID NO: 84 - DNA, 35 bp, Artificial
  synthetic
gggcaaggccgtgcaccaggtgttcggcaccgtgt

SEQ ID NO: 85 - DNA, 35 bp, Artificial
  synthetic
gccgctgaacatggcgccgtacacggtgccgaaca

SEQ ID NO: 86 - DNA, 35 bp, Artificial
  synthetic
cctggatcggcatgaacagccgcagcaccagcatg

SEQ ID NO: 87 - DNA, 35 bp, Artificial
  synthetic
acggcgatgcaggtcatgctcatgctggtgctgcg

SEQ ID NO: 88 - DNA, 35 bp, Artificial
  synthetic
gacgtgaagaagaagaacgtgatcggccgcctgat

SEQ ID NO: 89 - DNA, 35 bp, Artificial
  synthetic
cttcggcgacagctacatcgtgatcggcgtgggcg

SEQ ID NO: 90 - DNA, 35 bp, Artificial
  synthetic
gcaagatgttcgaggccaccgcccgcggcgccaag

SEQ ID NO: 91 - DNA, 35 bp, Artificial
  synthetic
ggcggcgtgttcaccagcctgggcaaggccgtgca

SEQ ID NO: 92 - DNA, 35 bp, Artificial
  synthetic
cctgatcggcatcctgatcacctggatcggcatga

SEQ ID NO: 93 - DNA, 35 bp, Artificial
  synthetic
cgaggccgagccccccttcggcgacagctacatcg

SEQ ID NO: 94 - DNA, 35 bp, Artificial
  synthetic
cccttcgagatcaaggacgtgaagaagaagaacgt

SEQ ID NO: 95 - DNA, 31 bp, Artificial
  synthetic
gtactggatccagatccccttcgagatcaag

SEQ ID NO: 96 - DNA, 35 bp, Artificial
  synthetic
gggggctcggcctcgatgttcacggggctgtcctt

SEQ ID NO: 97 - DNA, 35 bp, Artificial
  synthetic
cctcgaacatcttgccgatgctgctgcccttcttg

SEQ ID NO: 98 - DNA, 35 bp, Artificial
  synthetic
ggtgaacacgccgcccacgctgccgaagtcccagg

SEQ ID NO: 99 - DNA, 35 bp, Artificial
  synthetic
atcttcatggtccagctcacgccgctgaacatggc

SEQ ID NO: 100 - DNA, 35 bp, Artificial
  synthetic
ggtacagggtcacgatgcccacggcgatgcaggtc

SEQ ID NO: 101 - DNA, 35 bp, Artificial
  synthetic
aggatgccgatcaggatcttcatggtccagctcac

SEQ ID NO: 102 - DNA, 35 bp, Artificial
  synthetic
gcaccatcacgcccaggtacagggtcacgatgccc

SEQ ID NO: 103 - DNA, 31 bp, Artificial
  synthetic
actcgctcgagggcctgcaccatcacgccca